(19) United States
(12) Patent Application Publication
(10) Pub. No.: US 2010/0100099 A1
(43) Pub. Date: Apr. 22, 2010

(54) CURABLE MATERIAL TRANSFER AND DELIVERY DEVICE

(76) Inventors: Tayla Reilly, Chicago, IL (US); John Krueger, Muskego, WI (US); Jesse Darley, Madison, WI (US); Scott Biba, Highland, WI (US); Brian Ruffner, Antioch, IL (US); John Ray, Indian Creek, IL (US)

Correspondence Address: CareFusion Corp./BHGL, P.O. Box 10395, Chicago, IL, 60610, US

(21) Appl. No.: 12/489,231

(22) Filed: Jun. 22, 2009

Related U.S. Application Data
(60) Provisional application No. 61/075,204, filed on Jun. 24, 2008.

(51) Int. Cl. A61F 2/46; B01F 15/02; A61B 17/56; B67D 7/74

(52) U.S. Cl. 606/93; 366/183.1; 222/145.5; 222/391

(57) ABSTRACT

An apparatus and method for transferring curable material to an injector to convenient deliver the curable material to a patient. The apparatus contains a mixing chamber for mixing a liquid component and a powder component to form a curable material. The curable material is transferred to an injector to when the mixing chamber and injector are moved toward each other.

### CLAIM OF PRIORITY

This application claims the benefit, pursuant to 35 USC 119(e), of the earlier filing date of U.S. Provisional Patent Application Ser. No. 61/075,204, entitled “CURIBLE MATERIAL TRANSFER AND DELIVERY DEVICE,” filed in the U.S. Patent Office on Jun. 24, 2008, the contents of which are incorporated by reference, herein.

### 1. TECHNICAL FIELD

The present invention relates to devices and methods for delivering curable materials for use with stabilizing bone structures. More particularly, it relates to devices, systems and methods for delivering the curable materials.

### 2. BACKGROUND INFORMATION

Surgical intervention at damaged or compromised bone sites has proven highly beneficial for patients, for example patients with back pain associated with vertebral damage. Bones of the human skeletal system include mineralized tissue that can generally be categorized into two morphological groups: “cortical” bone and “cancellous” bone. Outer walls of all bones are composed of cortical bone, which has a dense, compact bone structure characterized by a microscopic porosity. Cancellous or “trabecular” bone forms the interior structure of bones. Cancellous bone is composed of a lattice of interconnected slender rods and plates known by the term “trabeculae.”

During certain bone procedures, cancellous bone is supplemented by an injection of a palliative (or curative) material employed to stabilize the trabeculae. For example, superior and inferior vertebrae in the spine can be beneficially stabilized by the injection of an appropriate, curable material (e.g., polymethylmethacrylate (PMMA) or other curable material). In other procedures, percutaneous injection under computed tomography (CT) and/or fluoroscopic guidance of stabilization material into vertebral compression fractures by, for example, transpedicular or parapedicular approaches, has proven beneficial in relieving pain and stabilizing damaged bone sites. Other skeletal bones (e.g., the femur) can be treated in a similar fashion. In any regard, bone in general, and cancellous bone in particular, can be strengthened and stabilized by a palliative injection of bone-compatible curable material.

The curable material used in the above procedures is typically fashioned by mixing a liquid component and a powder component within the operating room just prior to placement of the curable material into an injector wherein the injector is then used to introduce the curable material into the patient. Curable material may be prepared by mixing a very fine cement powder, typically PMMA, with a liquid monomer, typically methylmethacrylate.

According to some methods of the prior art, the components of the curable material are mixed in a mixing bowl and then transferred to a delivery system, such as a syringe or other injector, to deliver the curable material to the patient. This method can delay procedures while the cement is being transferred to the delivery system and the curable material may be spilled during the transfer. The delay increases procedure time and can cause the curable material to set before the procedure is completed. Additionally, the mixing of the components creates undesirable fumes that have an offensive odor to many.

According to other methods in the prior art, curable material delivery systems contain chambers for holding curable material prior to injection that possess cross-sectional areas that require significant force to drive the curable material from the chamber. Internal chamber pressures can typically be 1000 psi to 4000 psi or more. The required axial load to drive curable material from a chamber is equivalent to the chamber pressure multiplied by the cross sectional area of the chamber. As a result, chambers having a relatively large cross-sectional area create even higher axial load requirements on the injector device. Where an operator is manually introducing the force to inject the curable material, such higher force requirements can create operator discomfort during the injection procedure.

There exists a need in the medical device field for an improved curable material delivery device. The present invention provides an efficient device and method for mixing and delivering components of a curable material.

### BRIEF SUMMARY

In one embodiment, a device for dispensing curable material is provided. The device has a first housing having an interior surface defining a first chamber for holding curable material, the first chamber defining a cross-sectional area. The device also has a second housing having at least one opening and having an interior surface defining a second chamber for holding curable material, the second chamber defining a cross-sectional area that is smaller than the cross-sectional area of the first chamber. The device also has a plunger within the second chamber for applying force to dispense curable material from the second chamber wherein at least a portion of the second housing is operable to fit inside of the first chamber and the at least one opening is in fluid communication with the first chamber to receive curable material from the first chamber.

In another embodiment, a device for dispensing curable material is provided. The device has a mixing chamber having a volume of curable material, the mixing chamber defining a longitudinal axis and a cross-sectional area. The device also has an injector chamber having at least one opening and defining a longitudinal axis parallel to the longitudinal axis of the mixing chamber and defining a cross-sectional area that is smaller than the cross-sectional area of the first chamber wherein at least a portion of the injector chamber is operable to fit inside of the mixing chamber and the at least one opening is in fluid communication with the mixing chamber and is operable to receive curable material from the mixing chamber by driving the mixing chamber and the injector chamber together in the axial direction.

In yet another embodiment, a method of dispensing curable material from a chamber is provided. In one step, a first housing having an interior surface defining a first chamber and a cross-sectional area, the first chamber having a volume of curable material is provided. In another step a second housing is inserted into the first chamber through an opening in the first housing, the second chamber having at least one opening and having an interior surface defining a second chamber for holding curable material, the second chamber defining a cross-sectional area that is smaller than the cross-sectional area of the first chamber. In another step, a first housing and second housing are driven together to cause a volume of curable material to flow from the first chamber to the second chamber through the at least one opening in the second housing. In another step, a plunger is moved within the second chamber to engage the volume of curable material to dispense curable material from the second chamber.

In yet another embodiment, a method of preparing curable material is provided. In one step, the curable material is mixed in a mixing chamber having a longitudinal axis, the longitudinal axis of the mixing chamber being in the horizontal orientation during mixing. In another step, the longitudinal axis of the mixing chamber is oriented in the vertical orientation. In another step, curable material is transferred into the mixing chamber when the longitudinal axis of the mixing chamber is in the vertical orientation.

Advantages of the present invention will become more apparent to those skilled in the art from the following description of the preferred embodiments of the invention which have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1 is an exploded view of a mixer section according to a preferred embodiment of the present invention;

FIG. 2 is an exploded view of an injector according to a preferred embodiment of the present invention;

FIG. 3 is a cross-section view of a housing of an injector according to a preferred embodiment of the present invention;

FIG. 4A is a partial cross-section view of a mixer section and an injector prior to transferring curable material to the injector according to a preferred embodiment of the present invention;

FIG. 4B is a partial cross-section view of a mixer section and an injector after transferring curable material to the injector according to a preferred embodiment of the present invention;

FIG. 5 is an exploded view of an injector according to a preferred embodiment of the present invention;

FIG. 6A is a partial cross-section view of a mixer section and an injector prior to transferring curable material to the injector according to a preferred embodiment of the present invention;

FIG. 6B is a partial cross-section view of a mixer section and an injector during transferring curable material to the injector according to a preferred embodiment of the present invention;

FIG. 7A is a side view of an injector according to a preferred embodiment of the present invention;

FIG. 7B is a partial cross-section view of an injector according to a preferred embodiment of the present invention;

FIG. 8 is a partial cross-section view of an injector according to a preferred embodiment of the present invention;

FIG. 9A is a side view of a driver and injector in a horizontal orientation according to a preferred embodiment of the present invention; and

FIG. 9B is a side view of a driver and injector in a vertical orientation according to a preferred embodiment of the present invention.

### DETAILED DESCRIPTION OF THE DRAWINGS AND THE PRESENTLY PREFERRED EMBODIMENTS

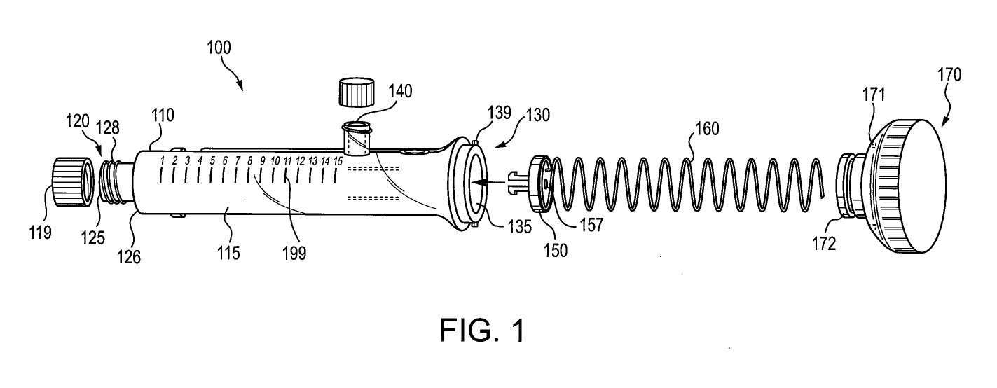

Details on the various components are provided below. In general terms, however, two separate components, preferably a liquid component and a powder component, are required to be mixed to form curable material for delivery to an injection site within a patient. FIG. 1 illustrates an embodiment of a mixer section 100 for a curable material transfer and delivery system according to principles of the present invention. Aspects of one embodiment of the mixer section 100 are described in more detail in U.S. application Ser. No. 11/890269, filed Aug. 3, 2007, the portions describing apparatuses for mixing curable material being incorporated herein by reference.

The mixer section 100, is highly useful for mixing a curable material. The phrase “curable material” within the context of the substance that can be delivered by the system/device of the invention described herein is intended to refer to materials (e.g., composites, polymers, and the like) that have a fluid or flowable state or phase and a hardened, solid or cured state or phase. Curable materials include, but are not limited to injectable bone cements (such as PMMA), which have a flowable state wherein they can be delivered (e.g., injected) by a cannula to a site and subsequently cure into hardened curable material. Other materials, such as calcium phosphates, bone in-growth material, antibiotics, proteins, etc., could be used to augment the curable material (but should not affect an overriding characteristic of the resultant formulation having a flowable state and a hardened, solid or cured state).

With reference to FIG. 1, a mixer section 100 according to one embodiment is disclosed. The mixer section 100 comprises a housing 110 that defines a mixing chamber 115. The housing 110 further comprises a first end 120 that has an opening 125 to the mixing chamber 115 and a second end 130 that has a second opening 135 to the mixing chamber 115. In one embodiment, the housing 110 also contains a port 140 that defines a passageway to the mixing chamber 115 for the introduction of the liquid component of the curable material.

According to the embodiment depicted in FIG. 1, the housing 110 is generally cylindrical and defines a longitudinal axis. The first end 120 and second end 130 are at opposite ends of the housing with respect to the longitudinal axis. According to a preferred embodiment, the first end 120 also has Luer-lock type threads 128 for mating with corresponding threads on a cap 119 or a delivery tube connector (not shown). The second end 130 preferably defines one or more injector locking features 139 that correspond to one or more openings 171 within a collar 170 (described in more detail below) so that the collar 170 may be removably connected with the housing 110. Although this embodiment uses injector locking features 139 to connect the housing 110 with the collar 170, one skilled in the art would know that other attachment means, such as a threaded connection or press-fit connection, may also be used.

The housing 110 is preferably transparent to provide the physician the ability to see the contents of the mixing chamber 115. In one embodiment, this allows the physician to see the progress of the mixing step of the components and to visually inspect the consistency of the curable material. The housing 110 is preferably made of nylon, but may also be made of cyclic olefin copolymer (COC), polycarbonate, Lexan®, and any other transparent material suitable for use with curable material, suitable for use at significant pressure, suitable to withstand sterilization and suitable to withstand gamma radiation without a substantial reduction in strength. With continued reference to FIG. 1, the housing 110 preferably also contains visual indicia 199 to indicate the volume of the curable material within the mixing chamber 115. The visual indicia 199 may be molded onto the housing 110, or may be painted or otherwise printed on the housing 110.

In one preferred embodiment, the diameter of mixing chamber is between about 0.5 inches and about 1 inch and the length of the mixing chamber is between about 2 inches and about 4 inches. These dimensions allow the mixing chamber to contain at least about 10 cc of curable material, which is a volume of curable material commonly used for injection into a delivery site.

In the embodiment of FIG. 1, the mixer section 100 also has mixing element holder 150 and a collapsible mixing element 160 for mixing the components of the curable material. The mixing element holder 150 connects to the collapsible mixing element 160 and both are located at least partially within the mixing chamber 115. The mixing element holder 150 further defines a passageway 157 that is operative to allow curable material to flow from within the mixing chamber 115 to outside the mixing chamber 115. The mixing element 160 can be rotated by engaging the mixing element holder 150 with a drive shaft from a motor (not shown) inserted through the first end 120. The drive shaft and the mixing element holder 150 interact so that rotation of the drive shaft rotates the mixing element holder 150 and thus, the collapsible mixing element 160.

According to a preferred embodiment depicted in FIG. 1, the mixer section 100 also comprises a removable collar 170 connected to the housing 110. In this embodiment, the collar 170 is removably connected with the second end 130 of the housing 110 and acts as cap on the housing 110 for transportation and storage and during mixing. The collar 170 contains a stopper 172 operative to seal the second end 130 of the housing 110. The stopper 172 preferably is substantially the same diameter of the mixing chamber 115 and forms a seal so that component material does not escape around the stopper 172. The mixing section also comprises a removable cap 119 that may be attached at the first end 120 of the housing 110 during transportation and storage.

Although the mixer section 100 has been described with reference to FIG. 1 above, chambers for mixing curable material may take other forms as well. As will be seen with respect to the disclosure of the injector devices herein, any chamber that is suitable for mixing or containing components of curable material that can engage the injector devices according to the embodiments of the present invention may be used. Chambers where mixing does not take place, but otherwise contains curable material, are also contemplated by the term “mixing chamber.”

The injector devices for injecting curable material according to the present invention are operable to receive curable material from the mixing chamber and dispense curable material into a delivery site. Generally, curable material is transferred from a curable material mixing chamber to an injector device by driving at least a portion of the injector device into the mixing chamber containing curable material. An opening in the injector allows the curable material to flow into a chamber of the injector device as the driving force is applied. The chamber of the injector for receiving the mixed curable material has a smaller cross-sectional area than the cross-sectional area of the mixing chamber. It has been observed that, during injection of the curable material to a delivery site, the relatively smaller cross-sectional area of the injector chamber creates a relatively low load requirement on the injector making injection of the curable material for the operator easier by requiring less actual and perceived effort.

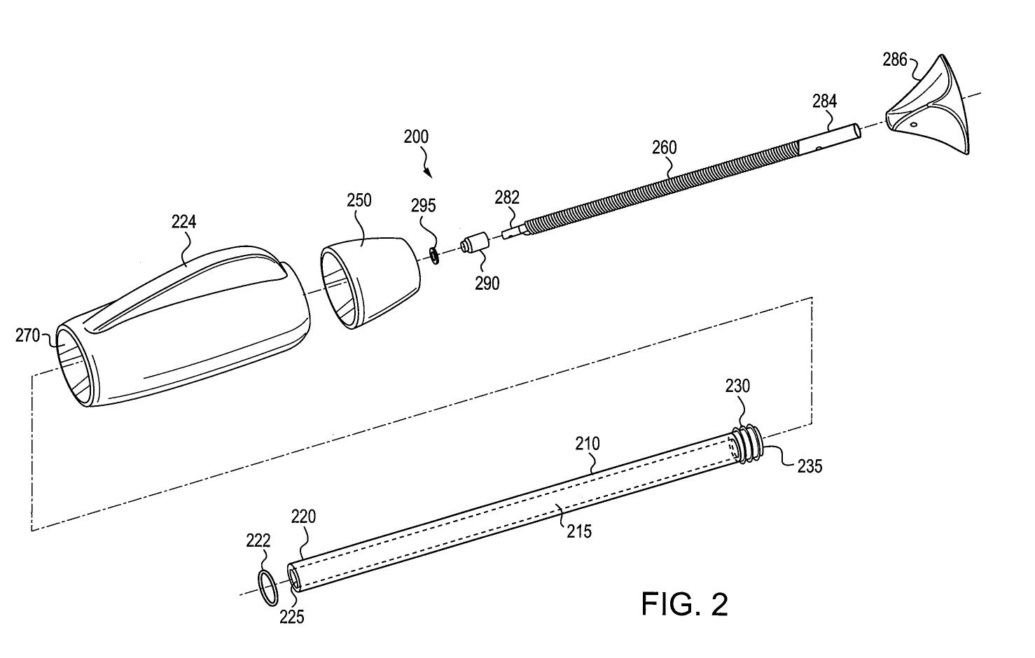

One embodiment of an injector 200 is shown in FIG. 2. With reference to FIG. 2, the injector 200 comprises a housing 210, an end body 250 and a force applicator in the form of a rod 260. The housing 210 defines a chamber 215, and further comprises a first end 220 that has a first opening 225 to the chamber 215 and a second end 230 that has a second opening 235 to the chamber. According to the embodiment depicted in FIG. 2, the housing 210 is generally cylindrical and defines a longitudinal axis. The first end 220 and second end 230 are at opposite ends of the housing with respect to the longitudinal axis.

In this embodiment, the injector chamber 215 has a cross-sectional area that is smaller than the cross-sectional area of the mixing chamber 115. Preferably, the volume of the injector chamber 215 is operable to be large enough to hold substantially the entire volume of curable material within the mixing chamber 115. With reference to FIG. 2, the injector housing 210 and injector chamber 215 are elongated relative to the mixer housing 110 and mixing chamber 115 to accommodate substantially the entire volume of curable material within the mixing chamber 115. In one preferred embodiment, the diameter of the injector chamber 215 is between about 0.2 inches and about 0.5 inches, and more preferably about 0.344 inches, and the length of the injector chamber is between about 3.7 inches and about 23.2 inches, and more preferably about 7.9 inches. Thus the cross sectional area is preferably between about 0.03 in² and about 0.2 in², and more preferably about 0.09 in². These dimensions allow the injector chamber 215 to contain at least about 10 cc of curable material, which is a volume of curable material commonly used for injection into a delivery site.

The injector housing 210 is preferably transparent to provide the physician the ability to see the contents of the mixing chamber 215. This will allow the physician to see the progress of the injection step. The injector housing 210 is preferably made of nylon, but may also be made of cyclic olefin copolymer (COC), polycarbonate, Lexan®, and any other transparent material suitable for use with curable material, suitable for use at significant pressure, suitable to withstand sterilization and suitable to withstand gamma radiation without a substantial reduction in strength.

The housing also preferably comprises an outer seal member 222 for engaging the interior wall defining the mixing chamber 115 of the mixer housing 110. In the embodiment of FIG. 2, the seal member 222 is located proximal to the first end 220 of the injector housing 210. The seal member 222 must be operable to withstand contact with curable material without decomposing and be operable to withstand pressures to form a seal with the inner surface of the mixer housing 110 without allowing curable material to leak behind the seal member 222 when a portion of the injector 200 is inserted into the mixing chamber 115. In one embodiment, with reference to FIG. 2, the outer seal 222 is a separate component such as an o-ring. In another embodiment, the outer seal 222 is integral with the injector 200. The outer seal 222 is preferably made of styrene-butadiene rubber (buna); however, other suitable materials, such as Polytetrafluoroethylene (PTFE) compounded with carbon fibers, may be used.

The injector housing 210, may also comprise a grip section 224 to allow a user to grip and more easily manipulate the injector 200. In the embodiment shown in FIG. 2, the grip section 224 is an enlarged cylindrical section located generally concentric to the injector housing 210. The grip section 224 may be fixedly attached to the injector housing 210, or may be formed integrally with the injector housing 210. One skilled in the art would understand that other configurations, such as a handle, may also be used to allow an operator to grip and more easily manipulate the injector 200, and that the grip section may not necessarily be attached to the injector housing 210 as long as the grip section 224 allows a user to grip and more easily manipulate the injector 200.

In the embodiment of FIG. 2, 4A-4B, the injector further comprises an end body 250 and a force applicator in the form of a threaded rod 260. The end body 250 is attached to the injector housing 210 at the second end 230 of the housing 210. Preferably, the end body 250 and injector housing 210 are connected with each other via a threaded connection; however, other connection methods would be known to one of skill in the art.

In another embodiment, the injector housing 210 is not threaded at the second end 230. In this embodiment, the second end 230 may be slightly enlarged to engage a recess within the grip section 224. The injector housing 210 is placed within the grip section 224 and the grip section 224 is connected to the end body 250 via a threaded connection, or other suitable connection. In this embodiment, the injector housing 210 is retained by the connection between the grip section 224 and the end body 250. Other connection methods may be used as well.

The end body 250 may also comprise an internal threaded portion (not shown) to engage the threaded rod 260. The threaded rod 260 contains a first end 282 proximal to a plunger 290. The threaded rod 260 also contains a second end 284 distal from the plunger 290 and having a handle 286. The threaded rod 260 and internal threaded portion of the end body 250 are operative so that when the handle 286 is turned, the threaded rod 260 moves axially in the direction of the first end 220 of the housing 210. As the threaded rod 260 moves axially, it advances the plunger 290 axially within the chamber 215 and thus applies force to inject curable material. In one preferred embodiment, the thread pitch of the threaded rod is 7 threads/inch; however other thread pitches may be used. One skilled in the art will understand that other manners of applying force to the curable material may also be used, such as a lever configuration or a ratchet-and-pawl configuration.

In one preferred embodiment of the plunger 290, the plunger 290 comprises a resilient member 295 to promote an adequate seal between the plunger 290 and the inner surface of the injector housing 210. The resilient member 295 must be operable to withstand contact with curable material without decomposing and be operable to withstand high pressures to form a seal with the inner surface of the housing without allowing curable material to leak behind the resilient member 295. In one embodiment, with reference to FIG. 2, the resilient member 295 is a separate component such as an o-ring. In another embodiment, the resilient member is integral with the plunger 290. The resilient member is preferably made of Polytetrafluoroethylene (PTFE) compounded with carbon fibers; however, other suitable materials may be used.

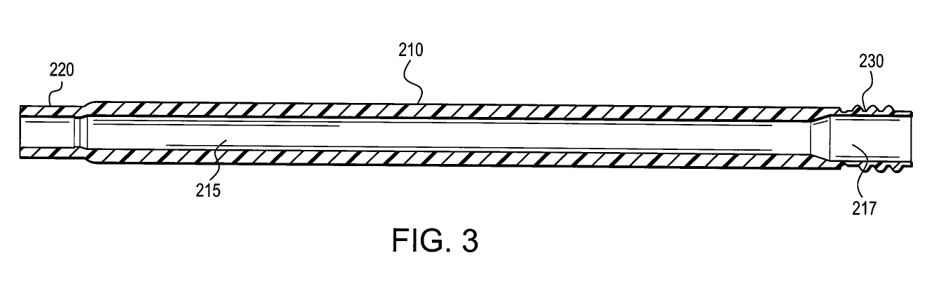

With reference to FIG. 3, in one embodiment of the injector 200, the chamber 215 of the housing 210 comprises a purging portion 217 proximal to the second end 230 of the housing 210. The inner cross-sectional area of the purging portion 217 is substantially larger than the cross-sectional area of the plunger 290 such that there is a clearance between the plunger 290 and the interior surface of the injector housing 210 at the purging portion 217 when the plunger 290 is within the purging portion 217. The clearance between the inner surface of the housing 210 and the plunger 290 allows gas to escape past the plunger 290 as the plunger 290 is advanced. This clearance also allows the plunger 290 to advance without a restrictive friction force between the inner surface of the housing 210 and the plunger 290. Alternatively, the purging portion 217 can define one or more shallow grooves. The one or more grooves are operative to allow air or other gas to travel around the plunger 290 as the plunger 290 advances through the chamber 215.

One or more vents (not shown) may be located within the injector 200 to allow gas to escape from the injector housing 210. As will be understood with reference to the operation of the device, gas within the chamber prior to transferring curable material into the chamber 215 will be allowed to escape through the vents as curable material flows into the chamber 215. The vents are preferably covered with a filter material so that gas escaping from the mixing chamber 215 has a reduced odor that is associated with the curable material. Preferably, the filter material is a Gore-tex® covering. Other filtering material, such as charcoal filtering material, may also be used.

In the embodiment shown in FIG. 2, the injector 200 also comprises a connector section 270 that engages the mixer section 100 to connect the mixer section 100 with the injector 200. With reference to FIGS. 1 and 2, the connector section 270 engages the one or more injector locking features 139 of the mixer section 100. Although this embodiment uses the injector locking features 139 to connect the housing 110 with the connector section 270, one skilled in the art would understand that other attachment means, such as a threaded connection or press-fit connection, may also be used. In one preferred embodiment, as shown in FIG. 2, the connector section 270 is formed integrally with the grip section 224.

In other embodiments, the connector section 270 may be separate from the grip section 224.

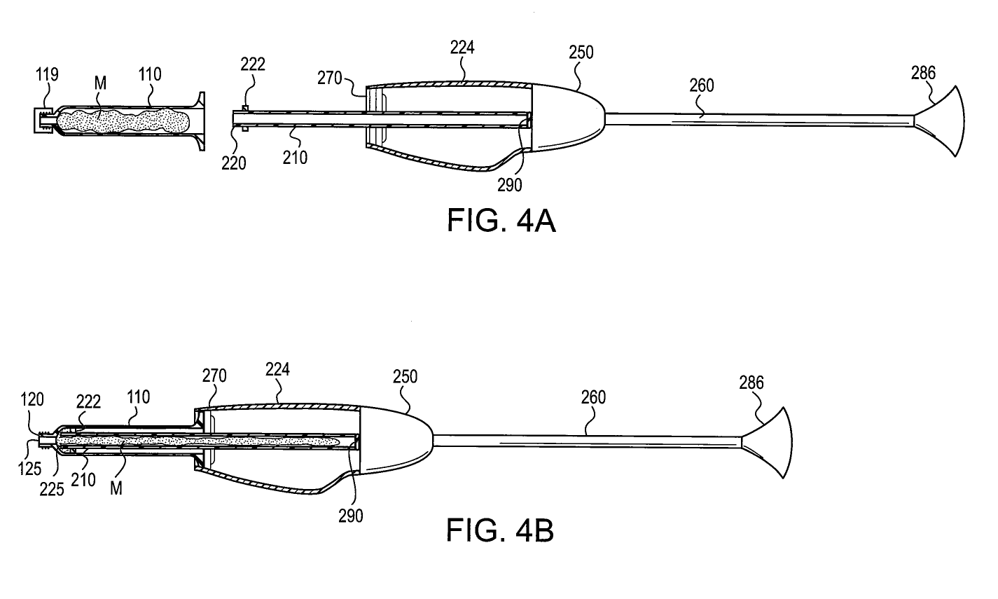

In operation, with reference to the embodiments in FIGS. 1-4B, curable material M is contained within the mixing chamber 115 of the mixer section 100. The operator then removes the end cap 170 from the second end 130 of the mixer housing 110. A portion of the injector housing 210 is then inserted into the mixing chamber 115 through the second end 130 of the mixer housing 110. The seal member 222 engages the inner wall of the mixer housing 110 to prevent curable material from flowing around the housing 210 of the injector 200 as it is pushed into the curable material in the mixing chamber 115. When the housing 210 of the injector is pushed into the curable material M within the mixing chamber 115, the curable material M is forced into the first opening 225 at the first end 220 of the housing 210. In this way, a significant volume of curable material M can be quickly transferred into the injector chamber 210 with relatively minimal effort by an operator. One skilled in the art will understand that the operator need only push the injector 200 and the mixing section 100 together such that at least a portion of the injector moves into the mixing chamber 115 in order to transfer curable material to the injector 200. Further, exposure to fumes from the curable material is minimized by the relatively quick transfer process and substantially closed system.

Additionally, the relatively smaller cross-sectional area of the injector chamber 215 requires reduced force input by an operator to inject curable material to a delivery site. For a cylindrical injector chamber having a diameter of about 0.344 inches, it has been observed that the torque at the handle of the injector required to achieve a chamber pressure of 2000 psi is less than 15 in-lb. By comparison, for a cylindrical injector chamber having a diameter of about 0.6875 inches, it has also been observed that the torque at the handle of the injector required to achieve a chamber pressure of 2000 psi is approximately 45 in-lb. Thus, a reduction in cross-sectional area of an injector chamber conveniently requires a reduced input from the operator.

In an embodiment of the mixer section 100 having a collapsible mixing element, the collapsible mixing element is compressed by the injector housing 210 as the injector 200 is driven into the curable material within the mixing chamber 115. As the first end of the housing 210 approaches the first end of the mixing section, curable material flows into the chamber and the collapsible mixing element becomes substantially compressed.

According to one embodiment, after the curable material has been transferred into the injector 200, the injector 200 and mixer section 100 are operable to be attached to each other such that at least a portion of the injector 200 remains inside of the mixing chamber 115 during injection of curable material. In one embodiment, the operator may connect mixer section 100 and the injector 200 via the connector section 270 and locking features 139. After connection, the opening 225 at the first end 220 of the housing 210 is aligned with the opening 125 in the first end 120 of the mixer housing 110 such that the openings are in fluid communication with each other. In an embodiment using a collapsible mixing element, the passageway 157 within the mixing element holder 150 is also in fluid communication with the openings 125, 225.

After curable material has been transferred into the injector 200, the operator removes the removable cap 119 from the mixer section 100. A delivery tube (not shown) may then be connected to the first end 120 of the mixer housing 110 to provide a lumen to a delivery site. The plunger 290 is advanced axially within the chamber 215 toward the first end 220 to drive curable material out of the injector chamber 215.

According to one embodiment, the mixed curable material does not occupy the entire volume of the injection chamber 215 after transfer. As a result, gas pockets exist within the injection chamber 215. As the plunger 210 is advanced within the injection chamber 215 toward the first end 220 of the housing 210, gas is allowed to escape through the purging portion 217 or through one or more grooves on the inner surface of the housing 210 toward the second end 230 of the housing 210 and rearward of the plunger 290. The purging portion 217 or grooves advantageously allow gas to be removed from the curable material as the plunger 290 advances and compresses the curable material. The removal of gas from the curable material beneficially provides a more consistent curable material and more efficient delivery of curable material.

In another embodiment, the injector 200 is not connected to the mixer section 100 during injection. In this embodiment, the injector 200 is removed from the mixing chamber 115 after the curable material is transferred to the injector 200 and the delivery tube is attached to the first end 220 of the injector 200. In this embodiment, the first end 220 of the housing 210 may contain a threaded connection to connect the injector 200 with the delivery tube, and the housing 210 may contain visual indicia to indicate the volume of the curable material within the injector chamber 215.

In another embodiment of the injector, multiple injectors having smaller volumes than the mixing chamber may be used. In this embodiment, only a portion of the curable material is transferred into a single injector during a curable material transfer step and, thus, multiple injectors may be used to deliver curable material to a delivery site.

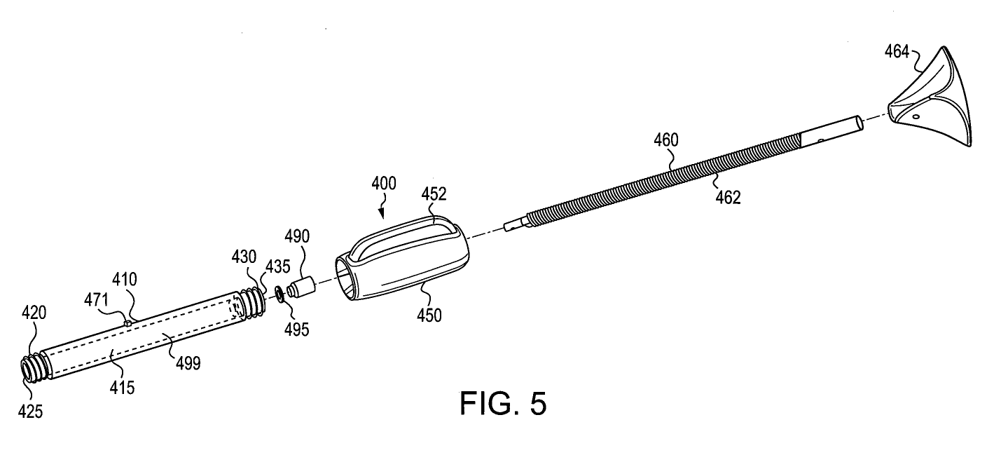

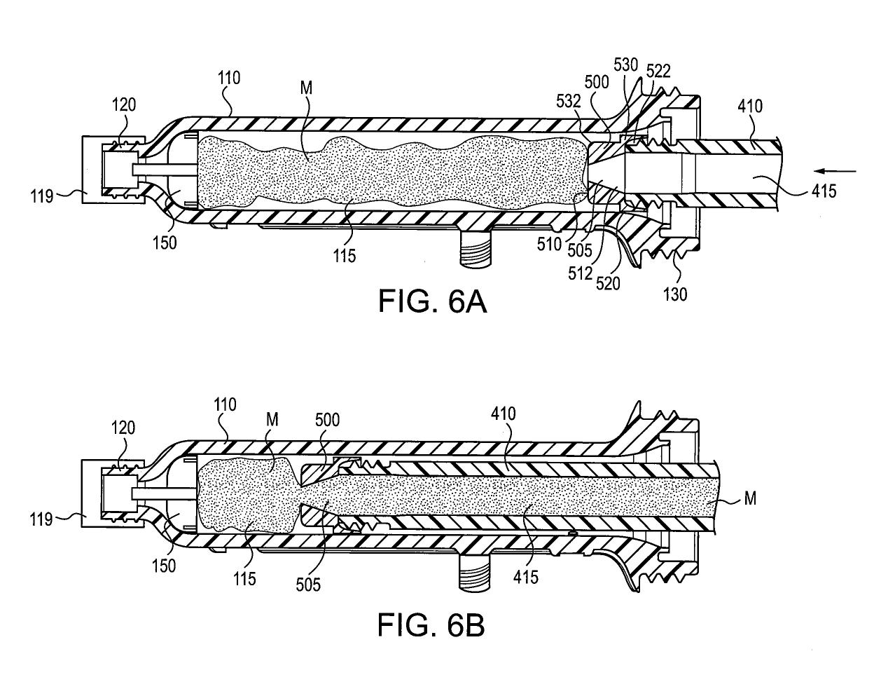

With reference to FIGS. 5-6B, one embodiment of an injector is disclosed. The injector 400 comprises a housing 410, a body 450 and a force applicator in the form of a rod 460. The housing 410 defines an injector chamber 415, and further comprises a first end 420 that has a first opening 425 to the chamber 415 and a second end 430 that has a second opening 435 to the chamber 415. According to the embodiment depicted in FIGS. 5-6B, the housing 410 is generally cylindrical and defines a longitudinal axis. The first end 420 and second end 430 are at opposite ends of the housing with respect to the longitudinal axis.

In this embodiment, the injector chamber 415 has cross-sectional area that is smaller than the cross-sectional area of the mixing chamber 115. The volume of the injector chamber 415 is operable to hold a portion of the volume of curable material within the mixing chamber 115. In this embodiment, the injector housing 410 and injector chamber 415 are not elongated relative to the embodiment shown in FIG. 2. The relatively compact size of the injector 400 promotes convenient and easy manipulation of the injector 400 during injection.

In one preferred embodiment, the diameter of the injector chamber is between about 0.2 inches and about 0.4 inches, and more preferably about 0.344 inches, and the length of the injector chamber is between about 3.4 inches and about 13.7 inches, and more preferably about 4.6 inches. Thus the cross sectional area is preferably between about 0.03 in² and about 0.13 in², and more preferably 0.09 in² These dimensions allow the injector chamber 115 to preferably contain approximately 5 cc to 7 cc of curable material.

The housing 410 preferably also contains visual indicia 499 to indicate the volume of the curable material within the injector chamber 415. The visual indicia 499 may be molded onto the housing 410, or may be painted or otherwise printed on the housing 410. The housing 410 is preferably transparent to provide the physician the ability to see the contents of the injector chamber 415. This will allow the physician to see the progress of the injection step. The housing 410 is preferably made of nylon, but may also be made of cyclic olefin copolymer (COC), polycarbonate, Lexan®, and any other transparent material suitable for use with curable material, suitable for use at significant pressure, suitable to withstand sterilization and suitable to withstand gamma radiation without a substantial reduction in strength.

In the embodiment of FIGS. 5-6B, the first end 420 has external threading for connection to a Luer-lock type of connector for a curable material delivery tube. Other known connecting mechanisms may be successfully interchanged, e.g., a conventional threaded hole, a thread and locking nut arrangement, etc. The second end 430 has external threading for connection of the housing 410 to the body 450. Other known connecting mechanisms may also be successfully interchanged

The injector body 450, assists in providing the application of force to drive curable material out of the injector chamber 415. According to one embodiment, the body 450 comprises an internal threaded portion (not shown) for engaging the threads 462 of the threaded rod 460. The threaded rod 460 has a plunger 490 at one end of the rod and a handle 464 at the opposite end of the rod 460. The plunger may include a resilient member 495 to provide a substantial seal between the inner surface of the injector housing 410 and the plunger 490. The threaded rod 460 and internal threaded section of the body 450 are operative so that when the handle 464 is turned, the threaded rod 460 moves axially in the direction of the first end 420 of the injector housing 410. As the threaded rod 460 moves axially, it advances the plunger 490 axially within the injector chamber 415. The body also preferably comprises an internal threaded portion (not shown) for engaging a threaded second end 430 of the injector housing 410. The body 450 further preferably comprises a grip section 452 to allow a physician to conveniently manipulate the body 450.

In one embodiment of the injector 400, the injector chamber 415 comprises a purging portion configured according to the purging portion 217 of FIG. 3.

With reference to FIGS. 6A-6B, the injector 400 of this embodiment also may engage a movable plug 500 located within the mixing chamber 115 of the mixing section 100. The movable plug 500 acts as an interface between the injector 400 and the mixing chamber 115 and promotes a convenient transfer of curable material from the mixing chamber 115 to one or more injectors 400. The movable plug 500 engages the inner surface of the mixing section 110 to substantially form a seal between the inner surface of the mixing section 110 and the movable plug 500. The movable plug 500 must be operable to withstand contact with curable material without decomposing. The plug 500 is preferably made of styrene-butadiene rubber (buna); however, other suitable materials, such as Polytetrafluoroethylene (PTFE) compounded with carbon fibers, may be used. The plug 500 must also be operable to move axially within the mixing chamber and still maintain a substantial seal.

With reference to FIGS. 6A-6B, the plug 500 has a first end 510, a second end 520, a lumen 505 between the first end 510 and second end 520, and a seal surface 530 for engaging the interior surface of the mixing chamber 115. The lumen 505 is operable to permit curable material to flow through the movable plug 500 when desired. In the embodiment of FIGS. 6A-6B, the first end 510 defines a tapered opening 512. The second end 520 defines an opening 522 operable to engage an end 420, 430 of the injector housing 410. At the beginning of the procedure, the plug is preferably located proximal to the second end 130 of the mixing section 100. In this way, as the movable plug 500 moves axially toward the first end 120 of the mixing chamber and contacts the curable material, the curable material will flow through the lumen 505 and into the injector 400. The plug 500 may reside in the mixing chamber 115 during mixing or may be inserted into the mixing chamber 115 after mixing. In one embodiment, the plug 500 can be removably connected with an end 420, 430 of the injector before being inserted into the mixing chamber 115. Insertion of the injector 400 into the mixing chamber therefore causes the plug 500 to be inserted into the mixing chamber 115. The plug 500 and injector 400 are removably connected with each other such that removal of the injector 400 from the mixing chamber 115 after the transfer of curable material will cause the injector 400 and movable plug 500 to separate and leave the plug 500 within the mixing chamber. Specifically with reference to the mixer section 100 of FIG. 1 and its collapsible mixing element 160, the plug 500 may also define a shoulder 532 that engages the collapsible mixing element 160 as the plug is moved within the mixing chamber 115.

With reference to the embodiments in FIGS. 1 and 5-6B, in operation, curable material is contained within the mixing chamber 115 of the mixer housing 110. The operator then removes the end cap 170 from the second end 130 of the mixer housing 110. A portion of the housing 410 of the injector 400 is inserted into the mixing chamber 115 through the second end 130 of the mixer housing 110 to engage the plug 500. In the embodiment shown in FIGS. 6A-6B, the second end 430 of the injector housing 410 engages the plug 500. The first end 420 of the injector housing 410 may also be connected in fluid communication with a delivery tube (not shown). When the housing 410 of the injector 400 and mixer housing 110 of the mixer section 100 are forced together, the injector 400 causes the plug 500 to come into contact with the curable material and the curable material is thus forced through the lumen 505 of the plug 500 and into the injector chamber 415. In this way, a significant volume of curable material can be quickly transferred into the injector housing 410 with relatively minimal effort by an operator. One skilled in the art will understand that the operator need only push, in one step, the injector housing 410 and the mixer housing 110 together in order to transfer curable material to the injector chamber 415. Further, exposure to fumes from the curable material is minimized by the relatively quick transfer process and substantially closed system. Also, in an embodiment where a delivery tube is connected with the first end 420 and the second end 430 is inserted into the mixing chamber 115, the clinician may continue to push the injector 400 such that the injector chamber 415 and delivery tube become filled with curable material, thus priming the delivery tube with curable material.

In another embodiment the first end 420 of the injector housing 410 may engage the plug 500. In this embodiment, the body 450 and threaded rod 460 may be attached to the second end 430 before or during transfer of curable material. In the embodiment where the second end 430 engages the plug 500 the delivery tube may be connected with the first end 420 before or during transfer of curable material.

In another embodiment, the injector 400 may also contain a stop member 471 that is operable to engage the mixer housing 110. In this embodiment, the stop member 471 allows the first end 420 of the injector 400 to be inserted into the mixing chamber 115 a desired distance, but then prevent further insertion. As a result, the transfer of curable material may be limited by the stop member 471 so that the injector 400 does not become overfilled.

In the embodiment of FIGS. 5-6B, the volume of the injector chamber 415 is less than the volume of curable material within the mixing chamber 115. As a result, the operator will cause the curable material to flow into the injector chamber 415 until the injector chamber 415 is filled. When filled, the operator removes the injector housing 410 from the mixing chamber 115. The operator then connects the injector housing 410 to the body 450 and rod 460 and advances the plunger 490 to force the curable material to be injected into the delivery site.

As will be understood by one of skill in the art, after the injector housing 410 is filled, the movable plug 500 remains inside of the mixing chamber 115 between the first end 120 and second end 130 of the mixer housing 110 and a volume of curable material remains in the mixer housing 110. The remaining curable material within the mixer housing 110 may be transferred in a second transfer process to an injector 400. More than two injectors 400 may be used as well, particularly if the injector chamber 415 in each injector 400 contains a relatively small volume, In one embodiment, the injector housing 410 is disconnected from the body 450 after curable material has been delivered to a delivery site and reinserted into the mixer housing 110. The injector housing 410 engages the plug 500 and further advances the plug 500 within the mixing chamber 115 to cause additional curable material to be transferred into the injector housing 410. In another embodiment, a second injector housing 410 is inserted into the mixing chamber according to the structures and procedures described herein to engage the plug 500 and further advances the plug 500 within the mixing chamber to cause additional curable material to be transferred into the second injector housing 410. After the transfer of additional curable material to the housing 410, additional curable material may be injected to a delivery site.

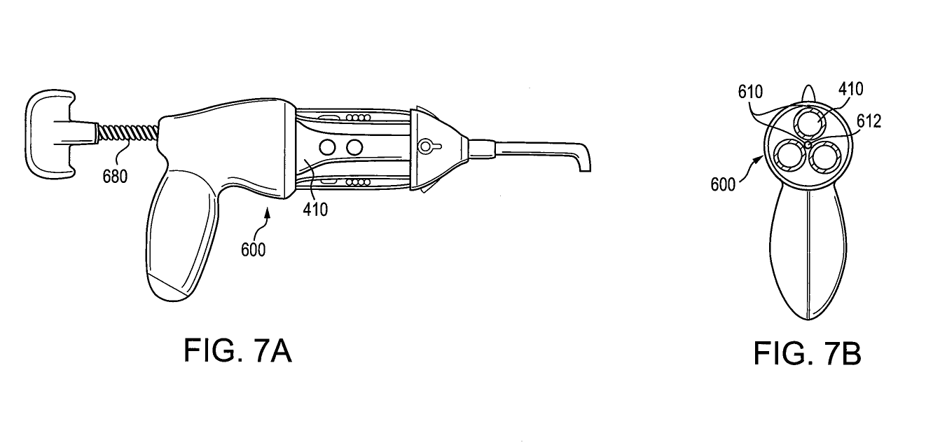

In another embodiment, a plurality of injector housings 410 may be used with a multi-barrel injector 600 operable to hold the plurality of injector housings 410. With reference to FIG. 7A, a multi-barrel injector 600 is shown comprising a revolving cartridge 610 for holding the plurality of injector housings 410. In this embodiment, and with reference to FIG. 7B, the cartridge 610 and injector housings 410 rotate around an axis 612. One of the injector housings 410 may be rotated such that its longitudinal axis is aligned with the axis of travel of a threaded rod 680. In this way, the advancement of the threaded rod 680 will force curable material to be dispensed from the injector housing 410. When desired, such as when one injector housing has been emptied of curable material, the cartridge 610 may be rotationally indexed, thus moving another injector housing 410 into alignment with the threaded rod 680. Curable material may then also be dispensed from this injector housing according to the procedures described above.

According to one embodiment, curable material may be transferred into the injector housings 410 according to the transfer procedures described above. In another embodiment, curable material may be prepared separately from the injector housing 410 and transferred in according to known methods before being connected with the multi-barrel injector 600. In another embodiment, curable material may be mixed in each injector housing 410 before being connected with the multi-barrel injector 600.

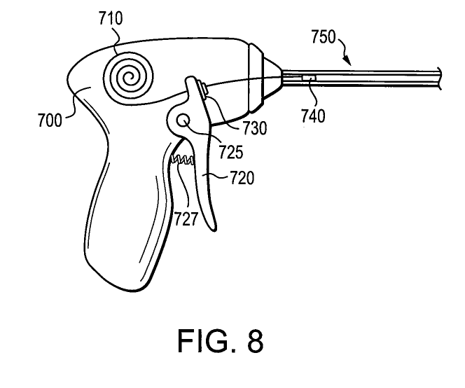

In another embodiment, the relatively small diameter injection chamber may be the delivery tube itself. With reference to FIG. 8, an injector 700 is shown having an elongated flexible rod 710 with a plunger 740. As can be seen in the figure, the flexible rod 710 is operable to be coiled to conveniently store the flexible rod 710 until use. According to one preferred embodiment, the flexible rod 710 is made of a braided wire such as stainless steel. The injector 700 also has a pivotal actuator 720 that rotates around a pivot 725 and a one way retainer 730. In this embodiment, the delivery tube 750 may act as the injection chamber for containing curable material itself; however, other relatively small diameter tubes may also be used.

In operation, the delivery tube 750 is filled with curable materials according to known methods or methods described herein. The injector 700 is then connected with the delivery tube 750. According to one embodiment, the clinician pulls on the pivotal actuator 720 causing the one way retainer 730 to rotate around the pivot 725. The one way retainer 730 then causes the flexible rod 710 to uncoil and advance in the direction of motion of the one way retainer 730. Upon releasing the pivotal actuator 720, a spring 727 applies force to the pivotal actuator 720 and causes it to pivot in the opposite direction. The one way retainer 730 is operable to allow the flexible rod 710 to slide through the one way retainer 730 as it pivots back to its original position. In this way, the flexible rod 710 remains in place and the one way retainer 730 is in position to advance the flexible rod 710 again when the pivotal actuator 720 is pulled.

According to one embodiment, the diameter of the delivery tube 750 is between about 0.16 inches and about 0.24 inches and the length is between about 17 inches to 38 inches. In this embodiment, the flexible tube may hold about 12.5 cc of curable material. The flexible rod 710 may also be between about 17 inches to 38 inches.

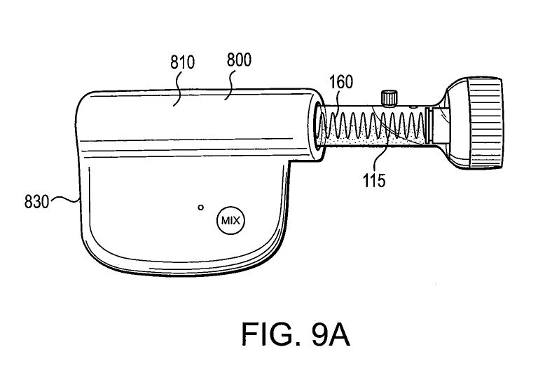

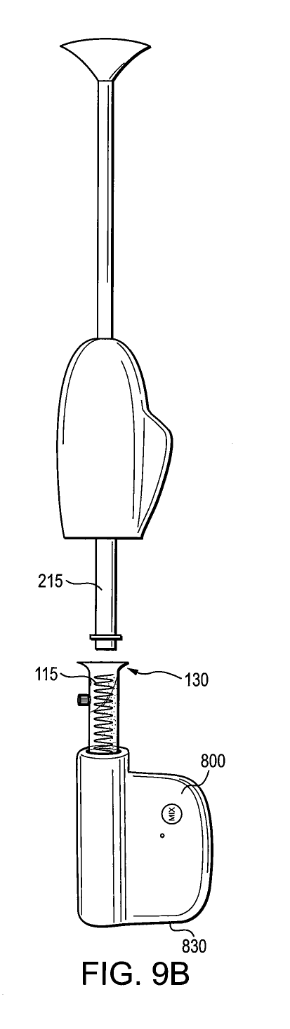

In another embodiment, a driver 800 for mixing the curable material may be used in the method of transferring the curable material to an injection chamber after mixing. In this embodiment, as depicted in FIG. 9A, a driver 800 having a driver housing 810 is shown. The driver 800 comprises a motor and a drive shaft for engaging the mixing element holder 150 within the mixing chamber 115. The mixing element 160 can be rotated by engaging the mixing element holder 150 with the drive shaft from a motor inserted through the first end 120 of the mixer section 100. The drive shaft and the mixing element holder 150 interact so that rotation of the drive shaft rotates the mixing element holder 150 and thus, the collapsible mixing element 160. It is preferable that the mixing chamber 115 is oriented horizontally with respect to the ground during mixing, as shown in FIG. 9A. Such an orientation has been observed to provide a consistent mixture. After mixing, the driver 800 and mixing chamber 115 are oriented vertically to facilitate transfer of curable material to an injector chamber. In this embodiment, the driver contains a base surface 830 that engages a substantially horizontal surface so that the driver 800 and mixing chamber 115 are substantially stabilized when placed in the vertical orientation with respect to the ground. In this way, the driver 800 acts as a base to the mixing chamber 115. The base surface 830 may be a substantially flat surface with a relatively large surface area, or the base surface 830 may have a plurality of legs that are wide enough apart from each other to allow the driver 800 to be substantially stable when oriented in the vertical orientation. The base surface 830, may also be weighted with weights to further stabilize the driver.

After the driver 800 and mixing chamber 115 are oriented vertically, the injection chamber 215 is also oriented substantially vertically and is translated along a vertical axis to be inserted into the mixing chamber 115 and cause curable material to be transferred to the injection chamber 215. Translation of the injector chamber 215 along the vertical axis allows for better control and convenience when transferring curable material.

In another embodiment, the collar 170, may be operable as a base to substantially stabilized the mixer housing 115 when placed in the vertical orientation with respect to the ground. In this embodiment, the collar contains a base surface and is operable to engage the first end 120 of the mixer housing 115. After mixing in the horizontal orientation, the clinician may remove the mixer housing from the driver 300. The clinician may also remove the collar and place the base surface on the substantially horizontal surface. The mixer housing 115 may then be connected with the collar and oriented in the vertical direction. In another embodiment, a separate base piece may be used to substantially stabilize the mixer housing 110 when placed in the vertical orientation with respect to the ground.

It is therefore intended that the foregoing detailed description be regarded as illustrative rather than limiting, and that it be understood that it is the following claims, including all equivalents, that are intended to define the spirit and scope of this invention.

What is claimed is:

1. A device for dispensing curable material comprising:
a first housing having an interior surface defining a first chamber for holding curable material, the first chamber defining a cross-sectional area;
a second housing having at least one opening and having an interior surface defining a second chamber for holding curable material, the second chamber defining a cross-sectional area that is smaller than the cross-sectional area of the first chamber, and
a plunger within the second chamber for applying force to dispense curable material from the second chamber wherein at least a portion of the second housing is operable to fit inside of the first chamber and the at least one opening is in fluid communication with the first chamber to receive curable material from the first chamber.

2. The device of claim 1 further comprising a seal member on the second housing operable to engage the interior surface of the first housing to provide a seal with the first chamber.

3. The device of claim 1 further comprising a connector for attaching the first housing to the second housing when curable material is dispensed from the second chamber.

4. The device of claim 1 wherein the cross-sectional area of the second chamber is between about 0.03 in² and about 0.2 in².

5. The device of claim 1 wherein the volume of the second chamber is at least about 10 cc.

6. The device of claim 1 further comprising a movable plug within the first chamber, the movable plug having a seal surface for sealing against the interior surface of the first chamber while allowing movement of the plug within the first chamber and having a lumen in fluid communication between the first chamber and the second chamber.

7. The device of claim 6 wherein an end of the second housing engages the movable plug.

8. The device of claim 6 wherein the cross-sectional area of the second chamber is between about 0.03 in² and about 0.13 in²

9. The device of claim 1 wherein the volume of the second chamber is less than about 7 cc and the device further comprises a third housing having at least one opening and having an interior surface defining a third chamber for holding curable material, the third chamber defining a cross-sectional area that is smaller than the cross-sectional area of the first chamber.

10. A device for dispensing curable material comprising:
a mixing chamber having a volume of curable material, the mixing chamber defining a longitudinal axis and a cross-sectional area;
an injector chamber having at least one opening and defining a longitudinal axis parallel to the longitudinal axis of the mixing chamber and defining a cross-sectional area that is smaller than the cross-sectional area of the first chamber wherein at least a portion of the injector chamber is operable to fit inside of the mixing chamber and the at least one opening is in fluid communication with the mixing chamber and is operable to receive curable material from the mixing chamber by driving the mixing chamber and the injector chamber together in the axial direction.

11. The device of claim 10 wherein the cross-sectional area of the second chamber is between about 0.03 in² and about 0.2 in².

12. The device of claim 10 wherein the volume of the second chamber is at least 10 cc.

13. The device of claim 10 further comprising a movable plug within the first chamber, the movable plug having a seal surface for sealing against the interior surface of the first chamber while allowing movement of the plug within the first chamber and having a lumen in fluid communication between the first chamber and the second chamber.

14. The device of claim 13 wherein the cross-sectional area of the second chamber is between about 0.03 in² and about 0.13 in².

15. The device of claim 10 wherein the volume of the second chamber is less than 6 cc and the device further comprises a second injector chamber having at least one opening and defining a longitudinal axis parallel to the longitudinal axis of the mixing chamber and defining a cross-sectional area that is smaller than the cross-sectional area of the mixing chamber.

16. A method of dispensing curable material from a chamber comprising the steps of:
providing a first housing having an interior surface defining a first chamber and a cross-sectional area, the first chamber having a volume of curable material;
inserting a second housing into the first chamber through an opening in the first housing, the second chamber having at least one opening and having an interior surface defining a second chamber for holding curable material, the second chamber defining a cross-sectional area that is smaller than the cross-sectional area of the first chamber,
driving the first housing and second housing together to cause a volume of curable material to flow from the first chamber to the second chamber through the at least one opening in the second housing, and
moving a plunger within the second chamber to engage the volume of curable material to dispense curable material from the second chamber.

17. The method of claim 16 further comprising attaching the first housing to the second housing prior to dispensing curable material from the second chamber.

18. The method of claim 16 further comprising a seal member on the second housing for providing a seal between the second housing and the interior surface of the first housing.

19. The method of claim 16 further comprising a movable plug having a lumen and a seal surface engaging the interior surface of the first chamber, the movable plug being located between a volume of curable material within the first chamber and the second housing wherein the second housing engages the movable plug when the first chamber and second chamber are driven together, causing the movable plug to move within the first chamber.

20. The device of claim 19 further comprising a third housing having at least one opening and having an interior surface defining a third chamber for holding curable material, the third chamber defining a cross-sectional area that is smaller than the cross-sectional area of the first chamber wherein a volume of curable material is transferred from the first chamber to the third chamber after the second chamber is removed from the first chamber. A method of preparing curable material comprising the steps of:
mixing the curable material in a mixing chamber having a longitudinal axis, the longitudinal axis of the mixing chamber being in the horizontal orientation during mixing;
orienting the longitudinal axis of the mixing chamber in the vertical orientation; and
transferring curable material into the mixing chamber when the longitudinal axis of the mixing chamber is in the vertical orientation.
